SEQUENCE LISTING (14 sequences)

SEQ ID NO: 1 - protein, 24 aa, synthetic construct
  source 1..24: note = HER2
LPESFDGDPASNTAPLQPEQLQVF

SEQ ID NO: 2 - protein, 21 aa, synthetic construct
  source 1..21: note = HER2
PESFDGDPASNTAPLQPEQLQ

SEQ ID NO: 3 - protein, 16 aa, synthetic construct
  source 1..16: note = CD38
QPEKVQTLEAWVIHGG

SEQ ID NO: 4 - protein, 15 aa, synthetic construct
  source 1..15: note = CD38
ISKRNIQFSCKNIYR

SEQ ID NO: 5 - protein, 10 aa, synthetic construct
  source 1..10: note = CD38
TFGSVEVHNL

SEQ ID NO: 6 - protein, 5 aa, synthetic construct
  source 1..5: note = CD38
QTLEA

SEQ ID NO: 7 - protein, 5 aa, synthetic construct
  source 1..5: note = CD38
IQFSC

SEQ ID NO: 8 - protein, 10 aa, synthetic construct
  source 1..10: note = PD-1
AFPEDRSQPG

SEQ ID NO: 9 - protein, 15 aa, synthetic construct
  source 1..15: note = peptide linker
TSGSGSGSGSGSGSG

SEQ ID NO: 10 - protein, 19 aa, synthetic construct
  source 1..19: note = amino acid sequence
ESFDGDPASNTAPLQPEQL

SEQ ID NO: 11 - RNA, 57 nt, synthetic construct
  source 1..57: mol_type = other RNA; note = mRNA sequence
gaatcatttgacggagatcccgctagtaacaccgcgccgctgcagccggagcagttg

SEQ ID NO: 12 - DNA, 85 bp, synthetic construct
  source 1..85: mol_type = other DNA; note = nucleic acid sequence
cctttagtggtacctttctattctcactctgctttcccggaagaccgttctcagccgggttcggccgaaactgttgaaagttgtt

SEQ ID NO: 13 - DNA, 85 bp, synthetic construct
  source 1..85: mol_type = other DNA; note = complementary nucleic acid sequence
aacaactttcaacagtttcggccgaacccggctgagaacggtcttccgggaaagcagagtgagaatagaaaggtaccactaaagg

SEQ ID NO: 14 - protein, 28 aa, synthetic construct
  source 1..28: note = construct
PLVVPFYSHSAFPEDRSQPGSAETVESC

(19) United States
(12) Patent Application Publication
(10) Pub. No.: US 2025/0144193 A1
(43) Pub. Date: May 8, 2025

(54) PROBIOTIC VACCINES AND RELATED METHODS OF USE

(71) Applicant: SALVITUS, INC., Colorado Springs, CO (US)

(72) Inventor: George PIECZENIK, Colorado Springs, CO (US)

(21) Appl. No.: 18/838,221

(22) PCT Filed: Feb. 21, 2023
(86) PCT No.: PCT/US2023/013542

§ 371 Date: Aug. 13, 2024

Related U.S. Application Data
(60) Provisional application No. 63/321,638, filed on Mar. 18, 2022.
(60) Provisional application No. 63/312,052, filed on Feb. 20, 2022.
(60) Provisional application No. 63/311,977, filed on Feb. 19, 2022.

(51) Int. Cl. A61K 39/00; C07K 14/47; C07K 14/705; C12N 15/73

(52) U.S. Cl. CPC A61K 39/001106; A61K 39/001111; A61K 39/001126; C07K 14/4748; C07K 14/70521; C12N 15/73; A61K 2039/523; C07K 2319/40; C12N 2795/00022; C12N 2795/00034; C12N 2795/00043

(57) ABSTRACT

Provided herein are probiotic vaccines comprising, a recombinant filamentous phage genome comprising a nucleic acid encoding a polypeptide comprising an exogenous peptide epitope, or fragments or variants thereof, selected from the group consisting of: LPESFDGDPASNTAPLQPEQLQVF “SEQ ID NO:1; HER2”. In particular embodiments of the invention vaccine, the exogenous peptide epitope is functionally expressed on a coat protein selected from the group consisting of: pill, pVI, pVII, pVIII and pIX. Also provided herein are methods of preventing or treating cancer, comprising administering to a patient in need thereof, one or more of the probiotic vaccines provided herein: or one or more of the recombinant phages provided herein

### RELATED APPLICATION

The present application claims priority to U.S. provisional patent application No. 63/311,977 filed Feb. 19, 2022; to U.S. provisional patent application No. 63/312,052 filed Feb. 20, 2022; and to U.S. provisional patent application No. 63/321,638 filed Mar. 18, 2022, each of which are incorporated herein by reference in its entirety.

### BACKGROUND

### Field of the Invention

The present invention relates recombinant phage, probiotic vaccines and methods of using same.

### Related Art

Cancer is more common today as a result of genetic abnormalities as well as epigenetic alterations in cell development and apoptotic pathways. Tumor development and growth are caused by the overexpression of genes associated with cell growth and proliferation in cancer and by the underexpression of genes that regulate cell death activities. Changes in cellular pathways are frequently produced by random mutations and unhealthy lifestyle habits. Immunocompetent individuals are vulnerable to natural immune alterations, which involve the activation of diverse immune responses to destroy competent tumor cells. Tumor-causing cells are going through apoptosis and necrosis as well as surrounding tissues are going through stress, and the microbiota in the gut all produce anti-cancer immune responses and signals.

Immunotherapy is a form of cancer treatment that activates the immune system to attack and eradicate cancer cells. Cytotoxic T lymphocytes (“CTL”) are critical to a successful antitumor immune response. T cells that attack cancer cells require the presentation of tumor antigens to naïve T cells that undergo activation, clonal expansion, and ultimately exert their cytolytic effector function. Effective antigen presentation is essential to successful CTL effector function. Thus, the development of a successful strategy to initiate presentation of tumor antigens to T cells can be important to an immunotherapeutic strategy for cancer treatment.

Likewise, B cells can inhibit tumor development through the production of tumor-reactive antibodies, promoting tumor killing by NK cells, phagocytosis by macrophages, and the priming of CD4+ and CD8+ T cells. B cells can promote tumor development through the production of autoantibodies and tumor growth factors.

With the clinical outcome of many types of cancers being from poor to lethal, there exists a significant need for the development of novel prophylactic and/or therapeutic treatments.

### SUMMARY

Provided herein are probiotic vaccines comprising,
a recombinant filamentous phage genome comprising a nucleic acid encoding a polypeptide comprising an exogenous peptide epitope, or fragments or variants thereof, selected from the group consisting of:

(SEQ ID NO: 1; HER2)
LPESFDGDPASNTAPLQPEQLQVF,
(SEQ ID NO: 2; HER2)
PESFDGDPASNTAPLQPEQLQ,
(SEQ ID NO: 3; CD38)
QPEKVQTLEAWVIHGG,
(SEQ ID NO: 4; CD38)
ISKRNIQFSCKNIYR,
(SEQ ID NO: 5; CD38)
TFGSVEVHNL,
(SEQ ID NO: 6; CD38)
QTLEA,
(SEQ ID NO: 7; CD38)
IQFSC,
and
(SEQ ID NO: 8; PD-1)
AFPEDRSQPG;

and
a bacterium infected with said recombinant phage.

In particular embodiments of the invention vaccine, the exogenous peptide epitope is functionally expressed on a coat protein selected from the group consisting of: pIII, pVI, pVII, pVIII and pIX. In a particular embodiment, the coat protein is pIII.

In further embodiments, the phage is selected from the group consisting of: filamentous phage, including, M13, fd, IKe, CTX-φ, Pfl, Pf2, Pf3, f1, MKE; M13K3; Myoviridae (Pl-like viruses; P2-like viruses; Mu-like viruses; SPOl-like viruses; phiH-like viruses); Siphoviridae (λ-like viruses, γ-like viruses, Tl-like viruses; T5-like viruses; c2-like viruses; L5-like viruses; psiMl-like viruses; phiC31-like viruses; N15-like viruses); Podoviridae (phi29-like viruses; P22-like viruses; N4-like viruses); Tectiviridae (Tectivirus); Corticoviridae (Corticovirus); Lipothrixviridae (Alphalipothrixvirus, Betalipothrixvirus, Gammalipothrixvirus, Deltalipothrixvirus); Plasmaviridae (Plasmavirus); Rudiviridae (Rudivirus); Fuselloviridae (Fusellovirus); Inoviridae (Inovirus, Plectrovirus, M13-like viruses, fd-like viruses); Microviridae (Microvirus, Spiromicrovirus, Bdellomicrovirus, Chlamydiamicrovirus); Leviviridae (Levivirus, Allolevivirus) and Cystoviridae (Cystovirus). In a particular embodiment, the phage is M13K3.

In yet other embodiments, the bacteria (e.g., probiotic bacteria) which is infected by the invention recombinant phage provided herein, can be selected from: E. coli Nissle 1917, E. coli ER2738, Bacillus amyloliquefaciens; Bacillus polyfermenticus, strain Bispan; Bifidobacterium animalis subsp. Lactis, strain BB-12; Bifidobacterium animalis subsp. Lactis, strain GPS1209; Bifidobacterium animalis subsp. Lactis, strain HN019 (DR1064); Bifidobacterium bifidum, strain BB-12; Bifidobacterium bifidum, strain Rosell-71; Bifidobacterium breve, strain M-16V; Bifidobacterium longum; Bifidobacterium thermophilum; Lactobacillus acidophilus, strain La-1; Lactobacillus brevis, strain HA-112; Lactobacillus fermentum, strain HA-179; Lactobacillus helveticus, strain Lafti L10; Lactobacillus helveticus, strain Rosell-52; Lactobacillus paracasei, strain Lafti L26; Lactobacillus paracasei subsp. paracasei, strain 431; Lactobacillus rhamnosus, strain HN001 (DR20); Streptococcus salivarius, strain DSM 13084; Streptococcus thermophilus; Bacillus coagulans GBI-30, 6086, Bifidobacterium animalis subsp. lactis BB-12, Bifidobacterium longum subsp. infantis, Escherichia coli Nissle 1917, E. coli ER2738, Lactobacillus acidophilus NCFM, Lactobacillus paracasei Stl 1 (or NCC2461), Lactobacillus johnsonii Lai (also referred to as Lactobacillus LCI, Lactobacillus johnsonii NCC533), Lactobacillus plantarum 299v, Lactobacillus reuteri ATCC 55730 (Lactobacillus reuteri SD2112), Lactobacillus reuteri Protectis (DSM 17938, daughter strain of ATCC 55730), Lactobacillus reuteri Prodentis (DSM 17938/ATCC 55730 and ATCC PTA 5289 in combination), Lactobacillus rhamnosus GG, Saccharomyces boulardii, mixture of Lactobacillus rhamnosus GR-1 and Lactobacillus reuteri RC-14, a mixture of Lactobacillus acidophilus NCFM and Bifidobacterium bifidum BB-12, a mixture of Lactobacillus acidophilus CL1285 and Lactobacillus casei LBC80R, a mixture of Lactobacillus plantarum HEAL 9 and Lactobacillus paracasei 8700:2, Lactobacillus bulgaricus, Lactococcus thermophiles and Lactobacillus bifidus. In a particular embodiment, the bacteria, which is infected by the invention recombinant phage provided herein, is F-factor positive. In another embodiment, the bacteria is F-factor positive selected from E. coli Nissle 1917 or E. coli ER2738. In a particular embodiment the bacteria is E. coli Nissle 1917. In another embodiment the bacteria, which is is E. coli ER2738.

In particular embodiments, the probiotic vaccine generates both IgG and IgA antibodies that bind to the exogenous peptide epitope. In other embodiments, the probiotic vaccine comprising the bacteria continually produces lysogenic phage. In yet further embodiments, of the probiotic vaccine, the peptide—TSGSGSGSGSGSGSG—is used as a linker between the coat protein and the exogenous peptide epitope.

Also provided herein is a recombinant filamentous phage genome comprising a recombinant phage genome comprising a nucleic acid encoding a polypeptide comprising an exogenous peptide epitope, or fragments or variants thereof, selected from the group consisting of:

(SEQ ID NO: 1; HER2)
LPESFDGDPASNTAPLQPEQLQVF,
(SEQ ID NO: 2; HER2)
PESFDGDPASNTAPLQPEQLQ,
(SEQ ID NO: 3; CD38)
QPEKVQTLEAWVIHGG,
(SEQ ID NO: 4; CD38)
ISKRNIQFSCKNIYR,
(SEQ ID NO: 5; CD38)
TFGSVEVHNL,
(SEQ ID NO: 6; CD38)
QTLEA,
(SEQ ID NO: 7; CD38)
IQFSC,
and
(SEQ ID NO: 8; PD-1)
AFPEDRSQPG.

In particular embodiments of the recombinant phage, the exogenous peptide epitope is functionally expressed on a coat protein selected from the group consisting of: pIII, pVI, pVII, pVIII and pIX. In a particular embodiment, of the recombinant phage, the coat protein is pIII . In further embodiments, the phage is selected from the group consisting of: filamentous phage, including, M13, fd, IKe, CTX-q, Pfl, Pf2, Pf3, f1, MKE; M13K3; Myoviridae (Pl-like viruses; P2-like viruses; Mu-like viruses; SPOl-like viruses; phiH-like viruses); Siphoviridae (λ-like viruses, γ-like viruses, Tl-like viruses; T5-like viruses; c2-like viruses; L5-like viruses; psiMl-like viruses; phiC31-like viruses; N15-like viruses); Pooviridae (phi29-like viruses; P22-like viruses; N4-like viruses); Tectiviridae (Tectivirus); Corticoviridae (Corticovirus); Lipothrixviridae (Alphalipothrixvirus, Betalipothrixvirus, Gammalipothrixvirus, Deltalipothrixvirus); Plasmaviridae (Plasmavirus); Rudiviridae (Rudivirus); Fuselloviridae (Fusellovirus); Inoviridae (Inovirus, Plectrovirus, M13-like viruses, fd-like viruses); Microviridae (Microvirus, Spiromicrovirus, Bdellomicrovirus, Chlamydiamicrovirus); Leviviridae (Levivirus, Allolevivirus) and Cystoviridae (Cystovirus). In yet further embodiments, the phage is a filamentous phage selected from the group consisting of: M13, fd, IKe, CTX-φ, Pfl, Pf2, Pf3, f1, MKE, and M13K3. In a particularly preferred embodiment, the phage is M13K3. In yet other embodiments, the recombinant phage generates IgG antibodies that bind to the exogenous peptide epitope. In yet further embodiments of the recombinant phage, the peptide—TSGSGSGSGSGSGSG—is used as a linker between the coat protein and the exogenous peptide epitope.

Also provided herein are methods of preventing or treating cancer, comprising administering to a patient in need thereof, one or more of the probiotic vaccines provided herein; or one or more of the recombinant phages provided herein. In particular embodiments, the cancer is selected from the group consisting of: multiple myeloma, epithelial cancer, epithelial ovarian cancer, mucosal melanoma, non-small cell lung cancer, melanoma, head and neck cancer, renal cell cancer, Hodgkin's lymphoma, Cutaneous Squamous Cell Carcinoma, glioblastoma, esophageal cancer, gastric cancer, duodenal cancer, small intestinal cancer, appendiceal cancer, large bowel cancer, colon cancer, rectum cancer, colorectal cancer, anal cancer, pancreatic cancer, liver cancer, gallbladder cancer, spleen cancer, renal cancer, bladder cancer, prostate cancer, testicular cancer, uterine cancer, endometrial cancer, ovarian cancer, vaginal cancer, vulvar cancer, breast cancer, pulmonary cancer, thyroid cancer, thymus cancer, brain cancer, nervous system cancer, gliomas, oral cavity cancer, skin cancer, blood cancer, lymphomas, eye cancer, bone cancer, bone marrow cancer, muscle cancer, non-small cell lung cancer (NSCLC), head and neck squamous cell cancer (HNSCC), urothelial carcinoma, non-muscle invasive bladder cancer [NMIBC]), colon or rectal cancer, esophageal or certain gastroesophageal junction (GEJ) carcinomas, cervical cancer, renal cell carcinoma (RCC), advanced endometrial carcinoma, cutaneous squamous cell carcinoma (cSCC), and/or triple-negative breast cancer (TNBC), and the like. In a particular embodiment, the cancer is multiple myeloma.

Also provided herein is a kit comprising the probiotic vaccine of the present invention or the recombinant phage of the present invention, together with instructions for administration of the probiotic vaccine or the recombiant phage.

Other features and advantages of the present invention will become more readily apparent to those of ordinary skill in the art after reviewing the following detailed description and accompanying drawings.

### BRIEF DESCRIPTION OF THE DRAWINGS

FIG. 1 shows the nucleic acid sequence at the insertion site of the nucleic acid encoding SEQ ID NO:8 in Example 3 to form the construct M13KE-AFP10 RF I DNA.

### DETAILED DESCRIPTION

Provided herein are novel probiotic vaccines comprising
a recombinant phage (e.g., filamentous phage) genome comprising a nucleic acid encoding a polypeptide comprising an exogenous peptide epitope, or fragments or variants thereof, selected from the group consisting of:

(SEQ ID NO: 1; HER2)
LPESFDGDPASNTAPLQPEQLQVF,
(SEQ ID NO: 2; HER2)
PESFDGDPASNTAPLQPEQLQ,
(SEQ ID NO: 3; CD38)
QPEKVQTLEAWVIHGG,
(SEQ ID NO: 4; CD38)
ISKRNIQFSCKNIYR,
(SEQ ID NO: 5; CD38)
TFGSVEVHNL,
(SEQ ID NO: 6; CD38)
QTLEA,
(SEQ ID NO: 7; CD38)
IQFSC,
and
(SEQ ID NO: 8; PD-1)
AFPEDRSQPG;

and
a bacterium infected with said recombinant phage.

As used herein, the phrase “probiotic vaccine” refers to the combination of a probiotic bacterium infected with a recombinant phage.

As set forth herein, the invention probiotic vaccines are particularly useful in methods for treating and/or preventing cancer or infectious disease. Unlike traditional vaccines, probiotic-based vaccines do not require critical storage conditions and transportation. Traditional live vaccines usually demand lower storage temperatures before administration. In addition, traditional vaccinations are developed using attenuated strains of pathogenic bacteria, such as Salmonella, Mycobacterium, and Bacillus, which have a number of drawbacks, including the possibility of attenuated strains reverting to virulent forms inside the body. The attenuated forms of vaccines also result in eliciting additional immune response in individual, hence weakening the effect of vaccine. The survival rates under the harshly acidic conditions of the human gut are another issue that contributes to the failure of traditional vaccines as compared with probiotic oral vaccines. The traditional forms of vaccines are generally not able to survive the harsh acidic conditions, and, as a result, they fail to reach the inner mucosal layer. On the other hand, probiotics are well versed with the acidic environmental conditions. Because probiotics are acid-and bile-tolerant, they offer a good choice for developing vaccines.

As used herein the term “vaccine”, refers to a composition capable of stimulating the immune system of a living organism so that protection against a harmful antigen is provided, either through prophylaxis or through therapy. Preferably, a vaccine or a vaccine composition further comprises one or more immuno-adjuvant substances.

As used herein, the term “preventing”, “prevention”, “prophylaxis” or “prevent” generally means to avoid or minimize the onset or development of a disease or condition before its onset, while the term “treating, “treatment” or “treat” encompasses reducing, ameliorating or curing a disease (e.g., cancer, and the like) or condition (or symptoms of a disease or condition) after its onset. The term “preventing” encompasses “reducing the likelihood of occurrence of” or “reducing the likelihood of reoccurrence”.

An “effective amount” or “effective dose” as used herein is an amount which provides the desired effect. For therapeutic purposes, an effective amount is an amount sufficient to provide a beneficial or desired clinical result. The preferred effective amount for a given application can be easily determined by the skilled person taking into consideration, for example, the size, age, weight of the subject, the type of disease/disorder to be prevented or treated, and the amount of time since the disease/disorder began. In the context of the present invention, in terms of prevention or treatment, an effective amount of the composition is an amount that is sufficient to induce a humoral and/or cell-mediated immune response directed against the disease/disorder.

According to the different aspects and embodiments of the invention described herein, a “subject” or “host” preferably refers to a mammal, and most preferably to a human being. Said subject may have, been suspected of having, or be at risk of developing cancer and/or an infectious disease (e.g. via the Korean Fever Virus infection, and the like).

### Exogenous Peptide Epitope

As used herein, the phrase “exogenous peptide epitope” refers to any peptide or sequence that is not native or natural to the host phage strain being used to produce the invention recombinant phage. In other words, the exogenous peptide epitope, and the nucleic acid encoding it, is heterologous (i.e., foreign) to the peptide sequences of the particular phage strain being utilized. In several particular embodiments, the length of the exogenous peptdide epitopes can be in a range selected from the group consisting of: 4-20 amino acids, 4-25 amino acids, 5-20 amino acids, 5-25 amino acids, 5-30 amino acids, 5-35 amino acids, 5-40 amino acids, 5-45 amino acids and 5-50 amino acids. In a particular embodiment, the length of the exogenous peptdide epitopes is in a range of 5-20 amino acids.

In accordance with the present invention, an exogoneous peptide epitope (e.g., an antigenic peptide) for use in the recombinant phage and/or probiotic vaccines provided herein comprise or consist of an amino acid sequence as set forth in any one of SEQ ID NOs:1-8, or fragments or variants thereof, as follows:

(SEQ ID NO: 1; HER2)
LPESFDGDPASNTAPLQPEQLQVF,
(SEQ ID NO: 2; HER2)
PESFDGDPASNTAPLQPEQLQ,
(SEQ ID NO: 3; CD38)
QPEKVQTLEAWVIHGG,
(SEQ ID NO: 4; CD38)
ISKRNIQFSCKNIYR,
(SEQ ID NO: 5; CD38)
TFGSVEVHNL,
(SEQ ID NO: 6; CD38)
QTLEA,
(SEQ ID NO: 7; CD38)
IQFSC,
and
(SEQ ID NO: 8; PD-1)
AFPEDRSQPG.

The epitopes used for culturing the recombinant phage with the probiotic bacterial cells will depend on the type of cancer to be treated or prevented; or infectious disease to be prevented. For example, in an embodiment using a HER2 exogenous peptide epitope for the treatment of cancer, including breast, bladder, pancreatic, ovarian, and/or stomach cancer, and the like, at least one exogenous peptide epitope, or fragments or variants thereof, is selected from: LPESFDGDPASNTAPLQPEQLQVF (SEQ ID NO:1), or PESFDGDPASNTAPLQPEQLQ (SEQ ID NO:2). In another embodiment, the exogenous peptide epitope comprises: LPESFDGDPASNTAPLQPEQLQVF (SEQ ID NO:1), or PESFDGDPASNTAPLOPEQLQ (SEQ ID NO:2) with five or fewer, four or fewer, three or fewer, two or fewer, or one amino acid substitution(s) within SEQ ID NOs:1 or 2. Accordingly, provided herein are methods of preventing or treating cancer, including breast, bladder, pancreatic, ovarian, and/or stomach cancer, comprising administering, to a patient in need thereof, a recombinant phage or probiotic vaccine comprising at least one exogenous peptide epitope, or fragment or veriant thereof, selected from: LPESFDGDPASNTAPLQPEQLQVF (SEQ ID NO:1), or PESFDGDPASNTAPLQPEQLQ (SEQ ID NO:2).

In another embodiment using a CD38 exogenous peptide epitope for the treatment of cancer, including leukemia, lymphoma, myeloma, multiple myeloma, CLL, and the like, at least one exogenous peptide epitope, or fragments or variants thereof, is selected from: QPEKVQTLEAWVIHGG (SEQ ID NO:3), ISKRNIQFSCKNIYR (SEQ ID NO:4), TFGSVEVHNL (SEQ ID NO:5), QTLEA (SEQ ID NO:6), and IQFSC (SEQ ID NO:7). In another embodiment, the exogenous peptide epitope comprises: QPEKVQTLEAWVIHGG (SEQ ID NO:3), ISKRNIQFSCKNIYR (SEQ ID NO:4), TFGSVEVHNL (SEQ ID NO:5), QTLEA (SEQ ID NO:6), and IQFSC (SEQ ID NO:7) with five or fewer, four or fewer, three or fewer, two or fewer, or one amino acid substitution(s) within SEQ ID NOs:3-7. Accordingly, provided herein are methods of preventing or treating cancer, including leukemia, lymphoma, myeloma, multiple myeloma, and/or chronic lymphocytic leukemia (CLL), comprising administering, to a patient in need thereof, a recombinant phage or probiotic vaccine comprising at least one exogenous peptide epitope, or fragment or veriant thereof, selected from: QPEKVQTLEAWVIHGG (SEQ ID NO:3), ISKRNIQFSCKNIYR (SEQ ID NO:4), TFGSVEVHNL (SEQ ID NO:5), QTLEA (SEQ ID NO:6), and IQFSC (SEQ ID NO:7).

In another embodiment, it is contemplated herein that exogenous peptide epitopes, or fragments or variants thereof, selected from: QPEKVQTLEAWVIHGG (SEQ ID NO:3), ISKRNIQFSCKNIYR (SEQ ID NO:4), TFGSVEVHNL (SEQ ID NO:5), QTLEA (SEQ ID NO:6), and IQFSC (SEQ ID NO:7), are able to prevent infection by the Korean Fever Virus. Accordingly, provided herein is a method of preventing Korean Fever Virus infection, comprising administering, to a patient in need thereof, a recombinant phage or probiotic vaccine comprising at least one exogenous peptide epitope, or fragment or veriant thereof, selected from: QPEKVQTLEAWVIHGG (SEQ ID NO:3), ISKRNIQFSCKNIYR (SEQ ID NO:4), TFGSVEVHNL (SEQ ID NO:5), QTLEA (SEQ ID NO:6), and IQFSC (SEQ ID NO:7).

In another embodiment using a PD-1 exogenous peptide epitope for the treatment of cancer, at least one exogenous peptide epitope corresponding to AFPEDRSQPG (SEQ ID NO:8), or fragments or variants thereof, are able to treat or prevent PD-1 mediated cancers, such as for example, mucosal membrane cancers, gastric cancer, melanoma, non-small cell lung cancer (NSCLC), head and neck squamous cell cancer (HNSCC), urothelial carcinoma, non-muscle invasive bladder cancer [NMIBC]), colon or rectal cancer, esophageal or certain gastroesophageal junction (GEJ) carcinomas, cervical cancer, renal cell carcinoma (RCC), advanced endometrial carcinoma, cutaneous squamous cell carcinoma (cSCC), and/or triple-negative breast cancer (TNBC), and the like. In another embodiment, the exogenous peptide epitope comprises: AFPEDRSQPG (SEQ ID NO:8), with five or fewer, four or fewer, three or fewer, two or fewer, or one amino acid substitution(s) within SEQ ID NO:8. Accordingly, provided herein are methods of preventing or treating cancer, comprising administering, to a patient in need thereof, a recombinant phage or probiotic vaccine comprising at least one exogenous peptide epitope corresponding to AFPEDRSQPG (SEQ ID NO:8), or fragments or variants thereof; wherein cancer is selected from the group consisting of: gastric cancer, melanoma, non-small cell lung cancer (NSCLC), head and neck squamous cell cancer (HNSCC), urothelial carcinoma, non-muscle invasive bladder cancer [NMIBC]), colon or rectal cancer, esophageal or certain gastroesophageal junction (GEJ) carcinomas, cervical cancer, renal cell carcinoma (RCC), advanced endometrial carcinoma, cutaneous squamous cell carcinoma (cSCC), and/or triple-negative breast cancer (TNBC).

As used herein, the term “fragments”, or grammatical variations thereof, refers to a smaller peptide (e.g., subset of amino acids) relative to the complete reference peptide sequence from which it is derived. For example, the fragment can be 1, 2, 3, 4, 5, 6, 7, 8, 9, 10, 11, 12, 13, 14, 15, 16, 17, 18, 19, 20, or more amino acids smaller than the complete reference peptide.

As used herein, the phrase “variants thereof”, “variant”, or grammatical variations thereof, refers to a biologically active polypeptide having at least about 80% amino acid sequence identity with the reference sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80%, 85%, 90%, 91%, 92%, 93%, 94%, 95%, 96%, 97%, 98% and 99% amino acid sequence identity. In some embodiments, a variant will have at least about 85% amino acid sequence identity. In some embodiments, a variant will have at least about 90% amino acid sequence identity. In some embodiments, a variant will have at least about 95% amino acid sequence identity with the native sequence polypeptide.

As used herein, “Percent (%) amino acid sequence identity” and “homology” with respect to a peptide or polypeptide sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

In particular embodiments, amino acid substitutions may include but are not limited to the replacement of one amino acid in a polypeptide with another amino acid. Exemplary conservative substitutions are shown in Table 1. In other embodiments, conservative amino acid substitutions may be introduced into an peptide of interest and the products screened for a desired activity, for example, increased immunogenicity, or the like.

In some embodiments, the modifying can include introducing at least one conservative substitution into the peptide or protein, in which at least one property such as the size, shape or charge of the amino acid is conserved. A “conservative amino acid substitution” refers to substitution of a structurally and/or functionally similar amino acid that may be made without not substantially altering the function of a protein. An example of conservative substitution is the exchange of an amino acid in one of the following groups for another amino acid of the same group (U.S. Pat. No. 5,767,063; Kyte and Doolittle, J. Mol. Biol. 157: 105-132 (1982)):
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

For example, substitutions can be made by changing, e.g., Val to Leu; Ser to Thr; or Asp to Glu. Other substitutions can also be considered conservative, depending on the environment of the particular amino acid and its role in the three-dimensional structure of the protein. For example, when it is desired to alter the pKa of an amino acid side chain while retaining the size and structure of the side chain, Glu may be substituted by Gln, and Asp may be substituted by Asn.

In some embodiments, the amino acid residues selected for modification (e.g., replacement with another amino acid residue) are selected from the group consisting of: His, Glu, Asp, Cys, Lys and Tyr. In some embodiments, the amino acid residues selected for modification include His and Glu residues. These amino acid residues typically have pKa values (even in the whole-protein context) of between about pH 6 to about pH 8, and therefore possess high buffering capacities In some embodiments, the at least one conservative amino acid substitution is selected from the group consisting of His to Arg, Glu to Gln, Asp to Asn, Lys to Arg, and Tyr to Phe.

In some embodiments, at least one of the one or more amino acid modifications includes a substitution of an amino acid with an alanine residue. Substitution or replacement of amino acid residues having high buffering capacities with alanine residues can be advantageous in applications in which it is desirable to reduce the buffering capacity of the peptide or protein. It should be noted that Nor Leucine is not coded but modified after substitution.

### Bacterium

As set forth above, provided herein are invention probiotic vaccines comprising a recombinant phage and a bacterium infected with said recombinant phage. In particular embodiments, any bacteria with F-factor (F⁺ cell), i.e., sex factor, that produces pili and a sex pilus for conjugation with other bacteria is suitable for use herein. When an F+ cell conjugates/mates with an F− cell, the result is two F+ cells, both capable of transmitting the plasmid to other F− cells by conjugation. A pilus on the F+ cell interacts with the recipient cell allowing formation of a mating junction, the DNA is nicked on one strand, unwound and transferred to the recipient. Exemplary F− factor-containing bacteria, include Escherichia coli Nissle 1917, E. coli ER2738, and the like.

Accordingly in particular embodiments, examples of probiotic bacteria for use in accordance with the present invention include, without limitation, Escherichia coli Nissle 1917, E. coli ER2738, Bacillus coagulans GBI-30, 6086, Bifidobacterium animalis subsp. lactis BB-12, Bifidobacterium longum subsp. infantis, Lactobacillus acidophilus NCFM, Lactobacillus paracasei Stl 1 (or NCC2461), Lactobacillus johnsonii Lai (also referred to as Lactobacillus LCI, Lactobacillus johnsonii NCC533), Lactobacillus plantarum 299v, Lactobacillus reuteri ATCC 55730 (Lactobacillus reuteriSD2112), Lactobacillus reuteri Protectis (DSM 17938, daughter strain of ATCC 55730), Lactobacillus reuteri Prodentis (DSM 17938/ATCC 55730 and ATCC PTA 5289 in combination), Lactobacillus rhamnosus GG, Saccharomyces boulardii, mixture of Lactobacillus rhamnosus GR-1 and Lactobacillus reuteri RC-14, a mixture of Lactobacillus acidophilus NCFM and Bifidobacterium bifidum BB-12, a mixture of Lactobacillus acidophilus CL1285 and Lactobacillus casei LBC80R, a mixture of Lactobacillus plantarum HEAL 9 and Lactobacillus paracasei 8700:2, Lactobacillus bulgaricus, Lactococcus thermophiles and/or Lactobacillus bifidus. In some embodiments, probiotic bacteria for use in accordance with the present disclosure may be a mixture of any two or more of the foregoing strains set forth herein. In particular embodiments, the probiotic bacteria is Escherichia coli Nissle 1917 or E. coli ER2738. In another embodiment, the probiotic bacteria is Escherichia coli Nissle 1917. In yet another embodiment, the probiotic bacteria is E. coli ER2738.

In other embodiments, bacteria suitable for use herein are small (typical linear dimensions of around 1 micron), non-compartmentalized organisms, with at least one circular DNA chromosomes and ribosomes of 70S. As used herein, the term “bacteria” encompasses all variants of bacteria (e.g., endogenous bacteria, which naturally reside in a closed system, environmental bacteria or bacteria released for bioremediation or other efforts).

In the invention probiotic vaccines provided herein, the invention recombinant phage are loaded (e.g., infected) into donor bacteria (e.g., probiotic bacteria and/or commensal bacteria) for delivery to to subjects in need thereof (e.g., human patients). Probiotic bacteria, for example, are live bacteria that can confer a health benefit on the host and/or, at the very least, are not harmful (e.g., not pathogenic) to the host, such as human patients. Thus, aspects of the invention contemplate the use of donor bacteria (e.g., probiotic bacteria and/or commensal bacteria) that is loaded or infected with non-lytic or inducible bacteriophage that can express and generate bacteriophage-based delivery particles in situ. Probiotics have also been found to penetrate the inner mucosal layer of the gut and aid in invasion of harmful bacteria, as well as protect against numerous enteric infections.

In particular embodiments, examples of probiotic bacteria for use herein for infection by the invention recombinant phage, include those set forth in the “PROBIO” database (“//bidd.group/probio/homepage.htm”; Shamekhi et al., Clin Transl Oncol (2020) 22(8):1227-39. doi:10.1007/s12094-019-02270-0; which is incorporated herein by reference in its entirety for all purposes), which database includes 329 probiotics that are currently commercially available, 115 probiotic bacteria that are undergoing clinical trials. In particular embodiments, the invention recombinant phage are infected or loaded into particular probiotic bacteria with immunomodulatory capabilities selected from the group consisting of: Bacillus amyloliquefaciens; Bacillus polyfermenticus, strain Bispan; Bifidobacterium animalis subsp. Lactis, strain BB-12; Bifidobacterium animalis subsp. Lactis, strain GPS1209; Bifidobacterium animalis subsp. Lactis, strain HN019 (DR1064); Bifidobacterium bifidum, strain BB-12; Bifidobacterium bifidum, strain Rosell-71; Bifidobacterium breve, strain M-16V; Bifidobacterium longum; Bifidobacterium thermophilum; Lactobacillus acidophilus, strain La-1; Lactobacillus brevis, strain HA-112; Lactobacillus fermentum, strain HA-179; Lactobacillus helveticus, strain Lafti L10; Lactobacillus helveticus, strain Rosell-52; Lactobacillus paracasei, strain Lafti L26; Lactobacillus paracasei subsp. paracasei, strain 431; Lactobacillus rhamnosus, strain HN001 (DR20); Streptococcus salivarius, strain DSM 13084; Streptococcus thermophilus. See, e.g., Table 1 of Singh et al., Front Immunol. 2022; 13: 1002674; Published online 2022 Oct. 3 doi: 10.3389/fimmu.2022.1002674; which is incorporated herein by reference in its entirety for all purposes.

In other embodiments, the invention recombinant bacteriophage of the present disclosure are contemplated herein to target bacteria other than Escherichia coli, including, without limitation, Bacteroides thetaiotamicron (e.g., BI), B. fragilis (e.g., ATCC 51477-B1, B40-8, Bf-1), B. caccae (e.g., phiHSCOI), B. ovatus (e.g., phiHSC02), Clostridium difficile (e.g., phiC2, phiC5, phiC6, phiC8, phiCD119,phiCD27), Klebsiella pneumoniae (e.g., KP01K2, KI I, Kpn5, KP34, JDOOI), Staphylococcus aureus (e.g., phiNMI, 80alpha), Enterococcus faecalis (e.g., IME-EF1), Enterococcus faecium (e.g., ENB6, C33), and Pseudomonas aeruginosa (e.g., phiKMV, PAK-P1, LKD16, LKA1, delta, sigma-1, J-I).

Thus, the bacteriophage of the present disclosure may target (e.g., specifically target) a bacterial cell from any one or more of the foregoing genus and/or species of bacteria. Other bacterial cells and microbes may also be targeted.

As used herein, “endogenous” bacterial cells may refer to non-pathogenic bacteria that are part of a normal internal ecosystem such as bacterial flora.

In yet further embodiments, bacterial cells of the present disclosure are anaerobic bacterial cells (e.g., cells that do not require oxygen for growth). Anaerobic bacterial cells include facultative anaerobic cells such as, for example, Escherichia coli, Shewanella oneidensis and Listeria monocytogenes. Anaerobic bacterial cells also include obligate anaerobic cells such as, for example, Bacteroides and Clostridium species. In humans, for example, anaerobic bacterial cells are most commonly found in the gastrointestinal tract. Thus, the bacteriophage of the present disclosure may target (e.g., specifically target) anaerobic bacterial cells.

### Recombinant Bacteriophage

Also provided herein is a recombinant filamentous phage genome comprising a recombinant phage genome comprising a nucleic acid encoding a polypeptide comprising an exogenous peptide epitope, or fragments or variants thereof, selected from the group consisting of:

(SEQ ID NO: 1; HER2)
LPESFDGDPASNTAPLQPEQLQVF,
(SEQ ID NO: 2; HER2)
PESFDGDPASNTAPLQPEQLQ,
(SEQ ID NO: 3; CD38)
QPEKVQTLEAWVIHGG,
(SEQ ID NO: 4; CD38)
ISKRNIQFSCKNIYR,
(SEQ ID NO: 5; CD38)
TFGSVEVHNL,
(SEQ ID NO: 6; CD38)
QTLEA,
(SEQ ID NO: 7; CD38)
IQFSC,
and
(SEQ ID NO: 8; PD-1)
AFPEDRSQPG.

In particular embodiments of the recombinant phage, the exogenous peptide epitope is functionally expressed on a coat protein of the phage selected from the group consisting of: pIII , pVI, pVII, pVIII and pIX. In a particular embodiment of the recombinant phage, the coat protein is pIII . As used herein, the phrase “functionally expressed” refers to the expression of the exogenous peptide epitope, such that the epitope is displayed on the surface of the phage and can elicit an immune response.

In further embodiments, the phage is selected from the group consisting of: filamentous phage, including, M13, fd, IKe, CTX-q, Pfl, Pf2, Pf3, f1, MKE; M13K3; Myoviridae (Pl-like viruses; P2-like viruses; Mu-like viruses; SPOl-like viruses; phiH-like viruses); Siphoviridae (λ-like viruses, γ-like viruses, Tl-like viruses; T5-like viruses; c2-like viruses; L5-like viruses; psiMl-like viruses; phiC31-like viruses; N15-like viruses); Pooviridae (phi29-like viruses; P22-like viruses; N4-like viruses); Tectiviridae (Tectivirus); Corticoviridae (Corticovirus); Lipothrixviridae (Alphalipothrixvirus, Betalipothrixvirus, Gammalipothrixvirus, Deltalipothrixvirus); Plasmaviridae (Plasmavirus); Rudiviridae (Rudivirus); Fuselloviridae (Fusellovirus); Inoviridae (Inovirus, Plectrovirus, M13-like viruses, fd-like viruses); Microviridae (Microvirus, Spiromicrovirus, Bdellomicrovirus, Chlamydiamicrovirus); Leviviridae (Levivirus, Allolevivirus) and Cystoviridae (Cystovirus). In yet further embodiments, the phage is a filamentous phage selected from the group consisting of: M13, fd, IKe, CTX-φ, Pfl, Pf2, Pf3, f1, MKE, and M13K3. In a particularly preferred embodiment, the phage is M13K3. In yet other embodiments, the recombinant phage generates IgG antibodies that bind to the exogenous peptide epitope.

In particular embodiments of the present disclosure, nucleic acid encoding exogenous peptide epitopes is recombinantly combined into naturally-occurring, engineered (e.g., rationally engineered), or adaptively evolved bacteriophage for delivery to microbial cell populations, e.g., probiotic bacterial cells. A bacteriophage, or phage, is a virus that infects and replicates in bacteria. Bacteriophages are composed of proteins that encapsulate a DNA or RNA genome and may have relatively simple or elaborate structures. Their genomes may encode as few as four genes, and as many as hundreds of genes. Bacteriophages replicate within bacteria following the injection of their genome into the cytoplasm and do so using either a lytic cycle, which results in bacterial cell lysis, or a lysogenic (non-lytic) cycle, which leaves the bacterial cell intact.

The bacteriophages of the present disclosure are, in some embodiments, non-lytic (also referred to as lysogenic or temperate)/ As used herein, the phrase “bacteria continually produces lysogenic phage” refers to a non-lytic phage infection of a bacteria, e.g., a probiotic bacterium set forth herein, such that the recombinant lysogenic phage are actively secreted from infected cells in the absence of lysis. Exemplary lysogenic (non-lytic) phage include without limitation, filamentous phage such as, for example, f1, M13, fd, IKe, CTX-q, Pfl, Pf2 and Pf3. Thus, after phage delivery of an exogenous peptide epitope into a bacterial cell, the bacterial cell may remain viable and able to stably maintain expression of antigenic epitope. In some embodiments, lytic bacteriophage may be used as delivery vehicles. When used with phagemid systems, naturally lytic phage serve as cargo shuttles and do not inherently lyse target cells.

Examples of non-lytic or non-lysogenic bacteriophage for use in accordance with the present disclosure include, without limitation, those selected from the group consisting of: Myoviridae (Pl-like viruses; P2-like viruses; Mu-like viruses; SPOl-like viruses; phiH-like viruses); Siphoviridae (λ-like viruses, γ-like viruses, Tl-like viruses; T5-like viruses; c2-like viruses; L5-like viruses; psiMl-like viruses; phiC31-like viruses; N15-like viruses); Podoviridae (phi29-like viruses; P22-like viruses; N4-like viruses); Tectiviridae (Tectivirus); Corticoviridae (Corticovirus); Lipothrixviridae (Alphalipothrixvirus, Betalipothrixvirus, Gammalipothrixvirus, Deltalipothrixvirus); Plasmaviridae (Plasmavirus); Rudiviridae (Rudivirus); Fuselloviridae (Fusellovirus); Inoviridae (Inovirus, Plectrovirus, M13-like viruses, f1-like viruses, fd-like viruses); Microviridae (Microvirus, Spiromicrovirus, Bdellomicrovirus, Chlamydiamicrovirus); Leviviridae (Levivirus, Allolevivirus) and Cystoviridae (Cystovirus). Such phages may be naturally occurring or engineered phages. In some embodiments, the bacteriophage is a coliphage (e.g., infects Escherichia coli). Those of skill in the art will readily understand that other bacteriophage may be used in accordance with the present disclosure.

In particular embodiments, the bacteriophages used herein are filamentous phages. Filamentous phages constitute a large family of bacterial viruses that infect many Gram-negative bacteria. Suitable well-known filamentous phages include those that infect Escherichia coli, such as, for example, f1, M13, fd and Ike, and the like. Phages f1, M13, and fd are those that have so far been used for filamentous phage display. Their genomes are more than 98% identical and their gene products are interchangeable.

A unique aspect of filamentous phage assembly, in contrast to the assembly of many other bacteriophages, is that it is a secretory process. Incorporation of coat polypeptides into the growing phage occurs in the cytoplasmic membrane, and nascent phages are extruded from the cell as they assembly. The E. coli cell does not lyse in this process. It is well-known to those of skill in the art that the five viral coat proteins (pIII, pVI, pVII, pVIII and pIX) are inserted in the cytoplasmic membrane prior to their incorporation into phage particles. For example, the major part of pIII is translocated across the membrane into the periplasm, while its C-terminal hydrophobic tail anchors the protein in the membrane.

In another embodiment, the bacteriophage used to prepare an invention recombinant phage is an M13 bacteriophage. M13 is a filamentous bacteriophage of the family Inoviridae and is composed of circular single-stranded DNA. M13 phages are about 900 nm long and 6-7 nm in diameter with 5 proteins. The minor coat protein, P3, attaches to the receptor at the tip of the F pilus of an Escherichia coli host cell. Thus, in a particular embodiment, the invention probiotic vaccines and their use in methods for treating or preventing cancer comprise delivering to bacterial cells a invention recombinant M13 bacteriophage that is engineered to functionally express at least one exogenous peptide epitope in one of its coat proteins selected from the group consisting of pIII, pVI, pVII, pVIII and pIX. In a particular embodiment, the exogenous peptide epitope is functionally expressed in gene pIII of the M13 bacteriophage.

The bacteriophages for use herein can be isolated from any environment where bacteria exist. In some embodiments, the recombinant bacteriophages of the invention are isolated from (e.g., collected from, obtained from) stool or sewage, terrestrial or marine environments.

### THERAPEUTIC METHODS

### Cancer

Also provided here, are methods of preventing or treating cancer or infectious disease, comprising administering to a patient in need thereof, the invention probiotic vaccines set forth herein, or the invention recombinant phage set forth herein.

As used herein, the term “cancer”, or grammatical variations thereof, refers to a malignant neoplasm. In particular, the term “cancer” refers herein to any member of a class of diseases or disorders that are characterized by uncontrolled division of cells and the ability of these cells to invade other tissues, either by direct growth into adjacent tissue through invasion or by implantation into distant sites by metastasis. Metastasis is defined as the stage in which cancer cells are transported through the bloodstream or lymphatic system. In particular embodiments, the cancer is selected from the group consisting of: multiple myeloma, epithelial cancer, epithelial ovarian cancer, mucosal melanoma, non-small cell lung cancer, melanoma, head and neck cancer, renal cell cancer, Hodgkin's lymphoma, Cutaneous Squamous Cell Carcinoma, glioblastoma, esophageal cancer, gastric cancer, duodenal cancer, small intestinal cancer, appendiceal cancer, large bowel cancer, colon cancer, rectum cancer, colorectal cancer, anal cancer, pancreatic cancer, liver cancer, gallbladder cancer, spleen cancer, renal cancer, bladder cancer, prostate cancer, testicular cancer, uterine cancer, endometrial cancer, ovarian cancer, vaginal cancer, vulvar cancer, breast cancer, pulmonary cancer, thyroid cancer, thymus cancer, brain cancer, nervous system cancer, gliomas, oral cavity cancer, skin cancer, blood cancer, lymphomas, eye cancer, bone cancer, bone marrow cancer, muscle cancer, non-small cell lung cancer (NSCLC), head and neck squamous cell cancer (HNSCC), urothelial carcinoma, non-muscle invasive bladder cancer [NMIBC]), colon or rectal cancer, esophageal or certain gastroesophageal junction (GEJ) carcinomas, cervical cancer, renal cell carcinoma (RCC), advanced endometrial carcinoma, cutaneous squamous cell carcinoma (cSCC), and/or triple-negative breast cancer (TNBC), and the like. In a particular embodiment, the cancer is multiple myeloma.

In a particular embodiment, as set forth above, provided herein are methods of preventing or treating breast, bladder, pancreatic, ovarian, and/or stomach cancer, comprising administering, to a patient in need thereof, a recombinant phage or probiotic vaccine comprising at least one exogenous peptide epitope, or fragment or veriant thereof, selected from: LPESFDGDPASNTAPLQPEQLQVF (SEQ ID NO:1), or PESFDGDPASNTAPLQPEQLQ (SEQ ID NO:2).

In another embodiment, as set forth above, provided herein are methods of preventing or treating multiple myeloma, comprising administering, to a patient in need thereof, a recombinant phage or probiotic vaccine comprising at least one exogenous peptide epitope, or fragment or veriant thereof, selected from: QPEKVQTLEAWVIHGG (SEQ ID NO:3), ISKRNIQFSCKNIYR (SEQ ID NO:4), TFGSVEVHNL (SEQ ID NO:5), QTLEA (SEQ ID NO:6), and IQFSC (SEQ ID NO:7).

In another embodiment using a PD-1 exogenous peptide epitope for the treatment of cancer, at least one exogenous peptide epitope corresponding to AFPEDRSQPG (SEQ ID NO:8), or fragments or variants thereof, are able to treat or prevent PD-1 mediated cancers, such as for example, mucosal membrane cancers, gastric cancer, melanoma, non-small cell lung cancer (NSCLC), head and neck squamous cell cancer (HNSCC), urothelial carcinoma, non-muscle invasive bladder cancer [NMIBC]), colon or rectal cancer, esophageal or certain gastroesophageal junction (GEJ) carcinomas, cervical cancer, renal cell carcinoma (RCC), advanced endometrial carcinoma, cutaneous squamous cell carcinoma (cSCC), and/or triple-negative breast cancer (TNBC), and the like. In another embodiment, the exogenous peptide epitope comprises: AFPEDRSQPG (SEQ ID NO:8), with five or fewer, four or fewer, three or fewer, two or fewer, or one amino acid substitution(s) within SEQ ID NO:8. Accordingly, provided herein are methods of preventing or treating cancer, comprising administering, to a patient in need thereof, a recombinant phage or probiotic vaccine comprising at least one exogenous peptide epitope corresponding to AFPEDRSQPG (SEQ ID NO:8), or fragments or variants thereof; wherein cancer is selected from the group consisting of: gastric cancer, melanoma, non-small cell lung cancer (NSCLC), head and neck squamous cell cancer (HNSCC), urothelial carcinoma, non-muscle invasive bladder cancer [NMIBC]), colon or rectal cancer, esophageal or certain gastroesophageal junction (GEJ) carcinomas, cervical cancer, renal cell carcinoma (RCC), advanced endometrial carcinoma, cutaneous squamous cell carcinoma (cSCC), and/or triple-negative breast cancer (TNBC).

### Infectious Disease

Viral hemorrhagic fevers (VHF) are a group of clinically similar diseases that can be caused by enveloped RNA viruses primarily from the families Arenaviridae, Filoviridae, Hantaviridae, and Flaviviridae. Clinically, this group of diseases has in common fever, fatigue, dizziness, muscle aches, and other associated symptoms that can progress to vascular leakage, bleeding and multi-organ failure. See, e.g., Perdomo-Celis et al., Vaccines 2019, 7(1), 11; . . . doi.org/10.3390/vaccines7010011; which is incorporated herein by reference in its entirety for all purposes.

VHFs can be transmitted person-to-person through direct contact with contaminated body fluids or tissues. Most of the VHF are also zoonotic in nature, spreading through different mechanisms depending on each virus. Some of them are transmitted through the consumption of raw meat or fluids from infected animals, direct contact with rodents or bats, inhalation or contact with materials contaminated with rodent excreta. Others are vector-borne diseases, transmitted by bites of infected mosquitoes or ticks. These diseases are clinically dynamic, ranging from asymptomatic to severe disease and death, rapidly progressing through several stages in a few hours or days. The lack of approved specific therapy contributes to disease burden and unfavorable clinical outcomes.

In another embodiment, as set forth above, provided herein is a method of preventing Korean Fever Virus (e.g., Hantaviridae or hantavirus) infection and/or disease manifestation, such Viral hemorrhagic fevers (VHF), and the like, comprising administering, to a patient in need thereof, a recombinant phage or probiotic vaccine comprising at least one exogenous peptide epitope, or fragment or veriant thereof, selected from: QPEKVQTLEAWVIHGG (SEQ ID NO:3), ISKRNIQFSCKNIYR (SEQ ID NO:4), TFGSVEVHNL (SEQ ID NO:5), QTLEA (SEQ ID NO:6), and IQFSC (SEQ ID NO:7).

### Administration of Probiotic Vaccine

The invention probiotic vaccine can be delivered to a patient (e.g., a patient having a cancer) or test animal by any suitable delivery route. In some embodiments, live probiotic bacterial cells may be delivered in vivo via fermented dairy products and/or probiotic fortified foods. Exemplary delivery foods can be selected from: probiotic solution, gummy, pill, yogurt, pickled vegetables, fermented bean paste (e.g., tempeh, miso, doenjang), kefir, buttermilk or karnemelk, kimchi, pao cai, sauerkraut, soy sauce, zha cai). In other embodiments, the probiotic bacteria may be delivered in vivo as tablets, capsules, powders and/or sachets containing the bacteria in freeze dried form.

In other embodiments, the invention probiotic vaccine can be delivered to a patient via injection, infusion, inoculation, direct surgical delivery, or any combination thereof. In some embodiments, the probiotic vaccine is administered to a human in the deltoid region or axillary region. For example, the vaccine is administered into the axillary region as an intradermal injection. In other embodiments, the vaccine is administered intravenously.

An appropriate carrier for administering the cells can be selected by one of skill in the art by routine techniques. For example, the pharmaceutical carrier can be a buffered saline solution, e.g., cell culture media, and can include DMSO for preserving cell viability. In certain embodiments, the cells are administered in an infusible cryopreservation medium. The composition comprising the cells can include DMSO and hetastarch as cryoprotectants, Plasmalyte A and/or dextrose solutions and human serum albumin as a protein component.

The quantity of probiotic vaccine for administration to a patient as a cancer vaccine to effect the methods described herein and the most convenient route of such administration are based upon a variety of factors, as can the formulation of the vaccine itself. Some of these factors include the physical characteristics of the patient (e.g., age, weight, and sex), the physical characteristics of the tumor (e.g., location, size, rate of growth, and accessibility), and the extent to which other therapeutic methodologies (e.g., chemotherapy, and beam radiation therapy) are being implemented in connection with an overall treatment regimen. Notwithstanding the variety of factors to be considered for implementing the methods of the present invention to prevent or therapeutically treat cancer or infectious diseases, a mammal, preferably a human, can be administered with from about 5×10⁶ to about 2×10⁸ probiotic vaccine cells in from about 0.05 mL to about 2 mL solution (e.g., saline) in a single administration. In other embodiments, a mammal, preferably a human, can be administered with from about 1×10³ to about 1×10¹⁵, 1×10⁴ to about 1×10¹⁴, 1×10⁵ to about 1×10¹³, 1×10⁵ to about 1×10¹², 1×10⁵ to about 1×10¹¹, 1×10⁵ to about 1×10¹⁰ 1×10⁵ to about 1×10⁹, 1×10⁵ to about 1×10⁸, 1×10⁶ to about 1×10¹², 1×10⁶ to about 1×10¹¹, 1×10⁶ to about 1×10¹⁰, 1×10⁶ to about 1×10⁹, 1×10⁶ to about 1×10⁸, probiotic vaccine cells in from about 0.05 mL to about 2 mL solution (e.g., saline) in a single administration.

Additional administrations can be carried out, depending upon the above-described and other factors, such as the severity of tumor pathology In further embodiments, from about one to about five administrations of: about 1×10⁴/ml, about 1×10⁵/ml, about 1×10⁶/ml, about 1×10⁷/ml, about 1×10⁸/ml, about 1×10⁹/ml, about 1×10¹⁰/ml, about 1×10¹¹/ml, about 1×10¹²/ml, about 1×10¹³/ml, about 1×10¹⁴/ml, about 1×10¹⁵/ml, probiotic vaccine cells is performed at two-week intervals. In one embodiment, from about one to about five administrations of about 1×10⁸/ml probiotic vaccine cells is performed at two-week intervals.

### Combination Treatments

Probiotic vaccination provided herein can be combined with other treatments. For example, a patient receiving the invention probiotic vaccination can also be receiving chemotherapy, immuno-oncology therapy, radiation, and/or surgical therapy before, concurrently, or after the direct and/or probiotic vaccination. Chemotherapy is used to shrink and slow cancer growth. Chemotherapy is recommended for numerous cancers after the initial surgery for cancer; however, sometimes chemotherapy is given to shrink the cancer before surgery. The number of cycles of chemotherapy treatment depends on the stage of the disease. Chemotherapy may neutralize antitumor immune response generated through vaccine therapy. In addition, chemotherapy can be combined safely with immunotherapy, with possibly additive or synergistic effects, as long as combinations are designed rationally. Examples of chemotherapeutic agents that can be used in treatments of patients with cancer include, but are not limited to, carboplatin, cisplatin, cyclophosphamide, docetaxel, doxorubicin, etoposide, gemcitabine, oxaliplatin, paclitaxel, TAXOL™, topotecan, and vinorelbine. In some embodiments, a patient receiving probiotic vaccination has already received chemotherapy, radiation, and/or surgical treatment for the gynecological or peritoneal cancer. Immunotherapy (Immuno-oncology therapy) refers to creating, facilitating and/or modulating an immune response by causing the production of antibodies to selected targets, such as TNF, and the like targets. Immunotherapies for use in combination with the invention probiotic vaccines (or invention recombinant phage) include treatment with Keytruda® (pembrolizumab), Opdivo® (nivolumab), and the like.

In addition to, or separate from chemotherapeutic treatment, a patient receiving an invention probiotic vaccination can be treated with any other treatments that are beneficial for the particular cancer. For example, a patient having ovarian, fallopian tube or peritoneal cancer, can be treated prior to, concurrently, or after the invention probiotic vaccination with a COX-2 inhibitor, as described, e.g., in Yu and Akasaki, WO 2005/037995. In another embodiment, a patient receiving an invention probiotic vaccination can be treated with bevacizumab (Avastin®) prior to, concurrently, or after probiotic vaccination.

Also provided herein is a kit comprising the probiotic vaccine of the present invention or the recombinant phage of the present invention, together with instructions for administration of the probiotic vaccine or the recombiant phage.

### EXAMPLES

The following examples are provided to better illustrate the claimed disclosure and are not to be interpreted as limiting the scope of the disclosure. To the extent that specific materials are mentioned, it is merely for purposes of illustration and is not intended to limit the disclosure. One skilled in the art can develop equivalent means or reactants without the exercise of inventive capacity and without departing from the scope of the disclosure.

### MATERIALS AND METHODS

### Example 1—Construction of HER2 Epitope-Containing Probiotic Vaccine

Provided herein is a bacteriophage oral vaccine comprising a HER2 epitope functionally expressed on a M13KE phage that has infected Nissle 1917 E. coli bacterial cells. Specifically, HER2 epitopes are integrated into coat protein gene pIII of MKE, fl and/or M13 bacteriophage producing a vaccination to overcome immunological tolerance against HER2. The carrier acts as an adjuvant and improves stability and B cell presentation of the epitopes.

It has been found that antibodies to two separate regions from the N- and C-terminal end of the fragment exhibited the growth inhibition. Epitope mapping of the C-terminal antibodies revealed a 24 amino acid region (LPESFDGDPASNTAPLQPEQLQVF) with two distinct epitopes mediating efficient growth inhibition. The results indicate that antibodies directed towards this region of domain III of the HER2, distinct from the well-known monoclonal antibodies trastuzumab and pertuzumab, bind to the HER2 on living cells and exhibit growth inhibition.

In a particular embodiment of the invention provided herein, the P4378-394 B-cell epitope from the extracellular domain of HER2 (PESFDGDPASNTAPLQPEQLQ) was displayed through insertion into gene III (i.e., pIII ) of a filamentous phage. It has previously been found in preclinical studies that immunization with P4378-394 epitopes as a single epitope or multi-epitope formulations induced HER2 specific IgG antibodies with strong anti-tumor activity. Furthermore, a clinical phase I study using a multi-epitope vaccine, containing the P4378-394 epitope, indicated that the vaccine was safe, well tolerated, and effective in overcoming immunological tolerance to HER2.

The mRNA sequence encoding the—ESFDGDPASNTAPLQPEQL—amino acid sequence was inserted between pIII signal peptide of M13KE RF I DNA and pIII protein coding sequence to construct M13KE-HER2 RF I DNA, as set forth in Example 3 below.

### Preparation of M13KE Vector

E. coli ER2738 in logarithmic growth phase was infected with M13KE phage. After culture, the bacteria were collected by centrifugation and used to extract plasmids by Axygen MidiPrep plasmid extraction kit. M13KE RF I DNA was further purified and then loaded into an agarose gel for QC.

M13KE RF I DNA was digested by Kpn 1/Eag I and then separated by agarose gel electrophoresis.

### Construction of M13KE-HER2 Phages

HER2-epitope encoding DNA was synthesized, digested by Kpn 1/Eag I, and ligated with M13KE RF I DNA. The construct of HER2-M13KE RF I DNA was transformed into E. coli ER2738 competent cells. After resuscitation, they infected E. coli ER2738 in logarithmic growth phase and cultured overnight at 32° C., on TOP-Agar LB (containing IPTG/X-gal) plates. Results indicated that there were many blue spots on the plate with the transformation of M13KE-HER2 into E. coli ER2738, while no blue spot was observed in control group. Several blue spots were selected and validated by sequencing. Results showed all clones had a correct construct correspond to the mRNA sequence:

GAATCATTTGACGGAGATCCCGCTAGTAACACCGCGCCGCTGCAGCCG
GAGCAGTTG.

### Phage Preparation

E. coli ER2738 in logarithmic growth phase was infected with M13KE phages inserted by correct HER2 epitope sequences. After overnight culture, the culture supernatant was collected, added with glycerol of 10% final concentration, and stored at −20° C.

The above M13K3-HER2 phage is used to infect the Nissle 1917 E. coli, which has been identified as male and is used as a probiotic. This Nissle infected bacteria with the HER2 epitope expressing bacteriophage is then used as a probiotic oral vaccine for the patient to make anti-HER2 antibodies against his/her cancer (e.g., breast, bladder, pancreatic, ovarian, and/or stomach cancer). Probiotic phage (i.g., phage+E. coli) containing the above epitopes are produced. Oral administration of the M113KE-HER2 probiotic is contemplated herein to generate IgA and IgG antibodies in the patient; whereas injection of the phage alone (separate from the probiotic bacteria) can generate IgG in the patient with reactivity toward HER2 on the patient's human cancer cells.

### Example 2—Construction of CD38 Epitope-Containing Probiotic Vaccine

The mRNA sequence encoding one of the QPEKVQTLEAWVIHGG (SEQ ID NO:3), ISKRNIQFSCKNIYR (SEQ ID NO:4), TFGSVEVHNL (SEQ ID NO:5), QTLEA (SEQ ID NO:6), and/or IQFSC (SEQ ID NO:7) amino acid sequences is inserted between pIII signal peptide of M13KE RF I DNA and pIII protein coding sequence to construct M13KE-CD38 RF I DNA.

### Preparation of M13KE Vector

E. coli ER2738 in logarithmic growth phase is infected with M13KE phage. After culture, the bacteria is collected by centrifugation and used to extract plasmids by Axygen MidiPrep plasmid extraction kit. M13KE RF I DNA is further purified and then loaded into an agarose gel for QC.

M13KE RF I DNA is digested by Kpn 1/Eag I and then separated by agarose gel electrophoresis.

### Construction of M13KE-CD38 Phages

CD38-epitope encoding DNA (encoding QPEKVQTLEAWVIHGG (SEQ ID NO:3), ISKRNIQFSCKNIYR (SEQ ID NO:4), TFGSVEVHNL (SEQ ID NO:5), QTLEA (SEQ ID NO:6), and/or IQFSC (SEQ ID NO:7)) is synthesized, digested by Kpn 1/Eag I, and ligated with M13KE RF I DNA. The construct of HER2-M13KE RF I DNA is transformed into E. coli ER2738 competent cells. After resuscitation, they infect E. coli ER2738 in logarithmic growth phase and are cultured overnight at 32° C., on TOP-Agar LB (containing IPTG/X-gal) plates. The results indicate that there are many blue spots on the plate with the transformation of M13KE-CD38 into E. coli ER2738, while no blue spot is observed in the control group. Several blue spots are selected and validated by sequencing. Results show that all clones had a correct construct corresponding to the mRNA sequence encoding: QPEKVQTLEAWVIHGG (SEQ ID NO:3), ISKRNIQFSCKNIYR (SEQ ID NO:4), TFGSVEVHNL (SEQ ID NO:5), QTLEA (SEQ ID NO:6), and/or IQFSC (SEQ ID NO:7).

### Phage Preparation

E. coli ER2738 in logarithmic growth phase is infected with M13KE phage having the correct CD38 exogenous peptide epitope inserted therein. After overnight culture, the culture supernatant is collected; glycerol is added to produce a 10% final concentration, and stored at −20° C.

The above M13K3-CD38 phage is used to infect the Nissle 1917 E. coli, and is used as a probiotic. This Nissle infected bacteria with the CD38 epitope expressing bacteriophage is then used as a Probiotic oral vaccine for the patient to make anti-CD38 antibodies against his/her cancer (such as multiple myeloma). Probiotic phage (i.g., phage+E. coli) containing the above epitopes are produced. Oral administration of the M113KE-CD38 Probiotic is contemplated herein to generate both IgA and IgG anti-CD38 antibodies in the patient; whereas injection of the phage alone (separate from the probiotic bacteria) can generate IgG in the patient with reactivity toward CD38 on the patient's human cancer cells.

### Example 3—Construction of PD-1/AFP10 Epitope-Containing Probiotic Vaccine

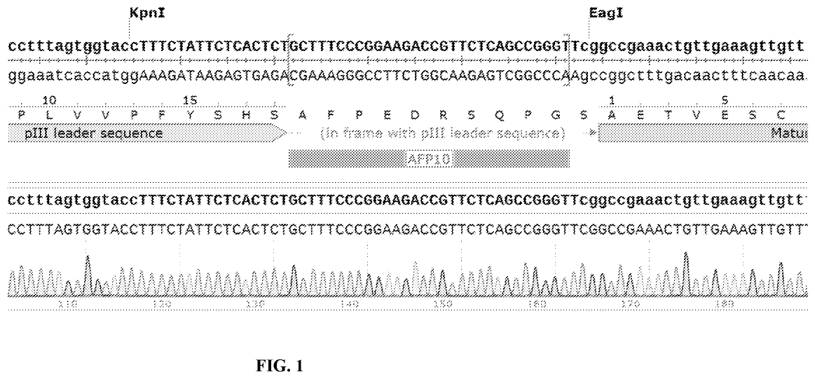

The mRNA sequence encoding the—AFPEDRSQPG—(SEQ ID NO:8; referred to herein as “AFP10”) amino acid sequence was inserted between pIII signal peptide of M13KE RF I DNA and pIII protein coding sequence to construct M13KE-AFP10 RF I DNA, and the sequence information is shown in FIG. 1.

### Preparation of M13KE Vector

E. coli ER2738 in logarithmic growth phase was infected with M13KE phage. After culture, the bacteria were collected by centrifugation and used to extract plasmids by Axygen MidiPrep plasmid extraction kit. M13KE RF I DNA was further purified and then loaded into an agarose gel for QC.

M13KE RF I DNA was digested by Kpn 1/Eag I and then separated by agarose gel electrophoresis.

### Construction of M13KE-AFP10 Phages

AFP10-epitope encoding DNA was synthesized, digested by Kpn 1/Eag I, and ligated with M13KE RF I DNA. The construct of AFP10-M13KE RF I DNA was transformed into E. coli ER2738 competent cells. After resuscitation, they infected E. coli ER2738 in logarithmic growth phase and cultured overnight at 32° C., on TOP-Agar LB (containing IPTG/X-gal) plates. Results indicated that there were many blue spots on the plate with the transformation of M13KE-AFP10 into E. coli ER2738, while no blue spot was observed in control group. Several blue spots were selected and validated by sequencing. Results showed all clones had a correct construct corresponding to the correct mRNA sequence.

### Phage Preparation

E. coli ER2738 in logarithmic growth phase was infected with M13KE phages having the correct AFP10 epitope sequence inserted therein. After overnight culture, the culture supernatant was collected, glycerol was added to produce a 10% final concentration, and it was stored at −20° C.

The above M13K3-AFP10 phage is used to infect the Nissle 1917 E. coli, and is used as a probiotic. This Nissle infected bacteria with the AFP10 epitope expressing bacteriophage is then used as a Probiotic oral vaccine for the patient to make anti-PD-1 antibodies against his/her cancer, such as gastric cancer, melanoma, non-small cell lung cancer (NSCLC), head and neck squamous cell cancer (HNSCC), urothelial carcinoma, non-muscle invasive bladder cancer [NMIBC]), colon or rectal cancer, esophageal or certain gastroesophageal junction (GEJ) carcinomas, cervical cancer, renal cell carcinoma (RCC), advanced endometrial carcinoma, cutaneous squamous cell carcinoma (cSCC), and/or triple-negative breast cancer (TNBC). Probiotic phage (i.e., phage+E. coli) containing the above epitopes are produced. Oral administration of the M113KE-AFP10 Probiotic is contemplated herein to generate both IgA and IgG anti-PD-1 antibodies in the patient; whereas injection of the phage alone (separate from the probiotic bacteria) can generate IgG in the patient with reactivity toward PD-1 on the patient's human cancer cells.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

1. A probiotic vaccine comprising,
a recombinant filamentous phage genome comprising a nucleic acid encoding a polypeptide comprising an exogenous peptide epitope, or fragments or variants thereof, selected from the group consisting of:
(SEQ ID NO: 1; HER2)
LPESFDGDPASNTAPLQPEQLQVF,
(SEQ ID NO: 2; HER2)
PESFDGDPASNTAPLQPEQLQ,
(SEQ ID NO: 3; CD38)
QPEKVQTLEAWVIHGG,
(SEQ ID NO: 4; CD38)
ISKRNIQFSCKNIYR,
(SEQ ID NO: 5; CD38)
TFGSVEVHNL,
(SEQ ID NO: 6; CD38)
QTLEA,
(SEQ ID NO: 7; CD38)
IQFSC,
and
(SEQ ID NO: 8; PD-1)
AFPEDRSQPG.
and
a bacteria infected with said recombinant phage.

2. The probiotic vaccine of claim 1, wherein the exogenous peptide epitope is functionally expressed on a coat protein selected from the group consisting of: pIII, pVI, pVII, pVIII and pIX.

3. The probiotic vaccine of claim 2, wherein the coat protein is pIII.

4. The probiotic vaccine of claim 1, wherein the phage is selected from the group consisting of: filamentous phage, including, M13, fd, IKe, CTX-φ, Pfl, Pf2, Pf3, f1, MKE; M13K3; Myoviridae (Pl-like viruses; P2-like viruses; Mu-like viruses; SPOl-like viruses; phiH-like viruses); Siphoviridae (λ-like viruses, γ-like viruses, Tl-like viruses; T5-like viruses; c2-like viruses; L5-like viruses; psiMl-like viruses; phiC31-like viruses; N15-like viruses); Podoviridae (phi29-like viruses; P22-like viruses; N4-like viruses); Tectiviridae (Tectivirus); Corticoviridae (Corticovirus); Lipothrixviridae (Alphalipothrixvirus, Betalipothrixvirus, Gammalipothrixvirus, Deltalipothrixvirus); Plasmaviridae (Plasmavirus); Rudiviridae (Rudivirus); Fuselloviridae (Fusellovirus); Inoviridae (Inovirus, Plectrovirus, M13-like viruses, fd-like viruses); Microviridae (Microvirus, Spiromicrovirus, Bdellomicrovirus, Chlamydiamicrovirus); Leviviridae (Levivirus, Allolevivirus) and Cystoviridae (Cystovirus).

5. The probiotic vaccine of claim 1, wherein the phage is M13K3.

6. The probiotic vaccine of claim 1, wherein the bacteria is selected from: E. coli Nissle 1917, E. coli ER2738, Bacillus amyloliquefaciens; Bacillus polyfermenticus, strain Bispan; Bifidobacterium animalis subsp. Lactis, strain BB-12; Bifidobacterium animalis subsp. Lactis, strain GPS1209; Bifidobacterium animalis subsp. Lactis, strain HN019 (DR1064); Bifidobacterium bifidum, strain BB-12; Bifidobacterium bifidum, strain Rosell-71; Bifidobacterium breve, strain M-16V; Bifidobacterium longum; Bifidobacterium thermophilum; Lactobacillus acidophilus, strain La-1; Lactobacillus brevis, strain HA-112; Lactobacillus fermentum, strain HA-179; Lactobacillus helveticus, strain Lafti L10; Lactobacillus helveticus, strain Rosell-52; Lactobacillus paracasei, strain Lafti L26; Lactobacillus paracasei subsp. paracasei, strain 431; Lactobacillus rhamnosus, strain HN001 (DR20); Streptococcus salivarius, strain DSM 13084; Streptococcus thermophilus; Bacillus coagulans GBI-30, 6086, Bifidobacterium animalis subsp. lactis BB-12, Bifidobacterium longum subsp. infantis, Escherichia coli Nissle 1917, Lactobacillus acidophilus NCFM, Lactobacillus paracasei Stl 1 (or NCC2461), Lactobacillus johnsonii Lai (also referred to as Lactobacillus LCI, Lactobacillus johnsonii NCC533), Lactobacillus plantarum 299v, Lactobacillus reuteri ATCC 55730 (Lactobacillus reuteri SD2112), Lactobacillus reuteri Protectis (DSM 17938, daughter strain of ATCC 55730), Lactobacillus reuteri Prodentis (DSM 17938/ATCC 55730 and ATCC PTA 5289 in combination), Lactobacillus rhamnosus GG, Saccharomyces boulardii, mixture of Lactobacillus rhamnosus GR-1 and Lactobacillus reuteri RC-14, a mixture of Lactobacillus acidophilus NCFM and Bifidobacterium bifidum BB-12, a mixture of Lactobacillus acidophilus CL1285 and Lactobacillus casei LBC80R, a mixture of Lactobacillus plantarum HEAL 9 and Lactobacillus paracasei 8700:2, Lactobacillus bulgaricus, Lactococcus thermophiles and Lactobacillus bifidus.

7. The probiotic vaccine of claim 1, wherein the bacteria is F-factor positive; and selected from E. coli Nissle 1917 or E. coli ER2738.

8. The probiotic vaccine of claim 1, wherein the probiotic vaccine generates both IgG and IgA antibodies that bind to the exogenous peptide epitope.

9. A recombinant filamentous phage genome comprising
a recombinant phage genome comprising a nucleic acid encoding a polypeptide comprising an exogenous peptide epitope, or fragments or variants thereof, selected from the group consisting of:
(SEQ ID NO: 1; HER2)
LPESFDGDPASNTAPLQPEQLQVF,
(SEQ ID NO: 2; HER2)
PESFDGDPASNTAPLQPEQLQ,
(SEQ ID NO: 3; CD38)
QPEKVQTLEAWVIHGG,
(SEQ ID NO: 4; CD38)
ISKRNIQFSCKNIYR,
(SEQ ID NO: 5; CD38)
TFGSVEVHNL,
(SEQ ID NO: 6; CD38)
QTLEA,
(SEQ ID NO: 7; CD38)
IQFSC,
and
(SEQ ID NO: 8; PD-1)
AFPEDRSQPG;

10. The recombinant phage of claim 9, wherein the exogenous peptide epitope is functionally expressed on a coat protein selected from the group consisting of: pIII, pVI, pVII, pVIII and pIX.

11. The recombinant phage of claim 10, wherein the coat protein is pIII.

12. The recombinant phage of claim 9, wherein the phage is selected from the group consisting of: filamentous phage, including, M13, fd, IKe, CTX-φ, Pfl, Pf2, Pf3, f1, MKE; M13K3; Myoviridae (Pl-like viruses; P2-like viruses; Mu-like viruses; SPOl-like viruses; phiH-like viruses); Siphoviridae (λ-like viruses, γ-like viruses, Tl-like viruses; T5-like viruses; c2-like viruses; L5-like viruses; psiMl-like viruses; phiC31-like viruses; N15-like viruses); Podoviridae (phi29-like viruses; P22-like viruses; N4-like viruses); Tectiviridae (Tectivirus); Corticoviridae (Corticovirus); Lipothrixviridae (Alphalipothrixvirus, Betalipothrixvirus, Gammalipothrixvirus, Deltalipothrixvirus); Plasmaviridae (Plasmavirus); Rudiviridae (Rudivirus); Fuselloviridae (Fusellovirus); Inoviridae (Inovirus, Plectrovirus, M13-like viruses, fd-like viruses); Microviridae (Microvirus, Spiromicrovirus, Bdellomicrovirus, Chlamydiamicrovirus); Leviviridae (Levivirus, Allolevivirus) and Cystoviridae (Cystovirus).

13. The recombinant phage of claim 9, wherein the phage is a filamentous phage selected from the group consisting of: M13, fd, IKe, CTX-φ, Pfl, Pf2, Pf3, f1, MKE, and M13K3.

14. The recombinant phage of claim 9, wherein the phage is M13K3.

15. The recombinant phage of claim 9, wherein the recombinant phage generates IgG antibodies that bind to the exogenous peptide epitope.

16. A method of preventing or treating cancer, comprising administering to a patient in need thereof, one or more of the probiotic vaccine of claims 1.

17. The method of claim 16, wherein the cancer is selected from the group consisting of: multiple myeloma, epithelial cancer, epithelial ovarian cancer, mucosal melanoma, non-small cell lung cancer, melanoma, head and neck cancer, renal cell cancer, Hodgkin's lymphoma, Cutaneous Squamous Cell Carcinoma, glioblastoma, esophageal cancer, gastric cancer, duodenal cancer, small intestinal cancer, appendiceal cancer, large bowel cancer, colon cancer, rectum cancer, colorectal cancer, anal cancer, pancreatic cancer, liver cancer, gallbladder cancer, spleen cancer, renal cancer, bladder cancer, prostate cancer, testicular cancer, uterine cancer, endometrial cancer, ovarian cancer, vaginal cancer, vulvar cancer, breast cancer, pulmonary cancer, thyroid cancer, thymus cancer, brain cancer, nervous system cancer, gliomas, oral cavity cancer, skin cancer, blood cancer, lymphomas, eye cancer, bone cancer, bone marrow cancer, muscle cancer, non-small cell lung cancer (NSCLC), head and neck squamous cell cancer (HNSCC), urothelial carcinoma, non-muscle invasive bladder cancer [NMIBC]), colon or rectal cancer, esophageal or certain gastroesophageal junction (GEJ) carcinomas, cervical cancer, renal cell carcinoma (RCC), advanced endometrial carcinoma, cutaneous squamous cell carcinoma (cSCC), and/or triple-negative breast cancer (TNBC).

18. The method of claim 16, wherein the cancer is multiple myeloma.

19. A method of preventing or treating breast, bladder, pancreatic, ovarian, and/or stomach cancer, comprising administering, to a patient in need thereof, a recombinant phage or probiotic vaccine comprising at least one exogenous peptide epitope, or fragment or veriant thereof, selected from: LPESFDGDPASNTAPLQPEQLQVF (SEQ ID NO:1), or PESFDGDPASNTAPLQPEQLQ (SEQ ID NO:2).

20. A method of preventing or treating leukemia, lymphoma, myeloma, multiple myeloma, and/or chronic lymphocytic leukemia (CLL), comprising administering, to a patient in need thereof, a recombinant phage or probiotic vaccine comprising at least one exogenous peptide epitope, or fragment or veriant thereof, selected from:
(SEQ ID NO: 3)
QPEKVQTLEAWVIHGG,
(SEQ ID NO: 4)
ISKRNIQFSCKNIYR,
(SEQ ID NO: 5)
TFGSVEVHNL,
(SEQ ID NO: 6)
QTLEA,
and
(SEQ ID NO: 7)
IQFSC.

21. A method of preventing Korean Fever Virus infection, comprising administering, to a patient in need thereof, a recombinant phage or probiotic vaccine comprising at least one exogenous peptide epitope, or fragment or veriant thereof, selected from: QPEKVQTLEAWVIHGG (SEQ ID NO:3), ISKRNIQFSCKNIYR (SEQ ID NO:4), TFGSVEVHNL (SEQ ID NO:5), QTLEA (SEQ ID NO:6), and IQFSC (SEQ ID NO:7).

22. A method of preventing or treating cancer, comprising administering, to a patient in need thereof, a recombinant phage or probiotic vaccine comprising at least one exogenous peptide epitope corresponding to AFPEDRSQPG (SEQ ID NO:8), or fragments or variants thereof; wherein cancer is selected from the group consisting of: gastric cancer, melanoma, non-small cell lung cancer (NSCLC), head and neck squamous cell cancer (HNSCC), urothelial carcinoma, non-muscle invasive bladder cancer [NMIBC]), colon or rectal cancer, esophageal or certain gastroesophageal junction (GEJ) carcinomas, cervical cancer, renal cell carcinoma (RCC), advanced endometrial carcinoma, cutaneous squamous cell carcinoma (cSCC), and/or triple-negative breast cancer (TNBC).

23. The probiotic vaccine of claim 1, wherein the bacteria continually produces lysogenic phage.

24. The probiotic phage of claim 1, or the recombinant phage of claims 9-15, wherein—TSGSGSGSGSGSGSG—is used as a linker between the coat protein and the exogenous peptide epitope.

25. A kit comprising the probiotic vaccine of claim 1 together with instructions for administration of the probiotic vaccine or the recombiant phage.
